(19) United States
(12) Patent Application Publication
(10) Pub. No.: US 2026/0162803 A1
(43) Pub. Date: Jun. 11, 2026

(54) AUTOMATED SWITCHING BETWEEN LOCAL AND CLOUD REPOSITORIES

(71) Applicant: SYNTHESIS HEALTH INC., Vancouver, BC (CA)

(72) Inventors: Jesse Wakley, Meridian, ID (US); Murray A. Reicher, Rancho Santa Fe, CA (US); Deepak Kaura, Calgary, Alberta (CA)

(21) Appl. No.: 19/123,708

(22) PCT Filed: Nov. 8, 2023
(86) PCT No.: PCT/US2023/079117

§ 371 Date: Apr. 23, 2025

Related U.S. Application Data
(60) Provisional application No. 63/382,950, filed on Nov. 9, 2022.

(51) Int. Cl. G16H 30/20; G16H 30/40

(52) U.S. Cl. CPC G16H 30/20; G16H 30/40

(57) ABSTRACT

A prefetching component on a user device (e.g., mobile or desktop computing system) can access a remote repository to prefetch one or more exams to a local data store based on one or more prefetching rules. A browser-based viewer program executes on the user device to implement an API that provides access to the locally stored exams, such as via a localhost server running on the computer system, so that images of the exams may be quickly and efficiently displayed in the viewer program. If the API is not available on the user device or if a requested exam has not been prefetched, the viewer program can initiate fetching of the exams from a remote repository for viewing in the viewer program.

### CROSS REFERENCE TO RELATED APPLICATIONS

This application claims benefit of priority to U.S. Provisional Application No. 63/382,950, filed Nov. 9, 2022, the disclosures of which is hereby incorporated herein in its entirety for all purposes.

### FIELD OF THE DISCLOSURE

The present disclosure relates to the field of medical imaging and patient healthcare.

### BACKGROUND

A variety of imaging modalities exist to produce medical images of physiological tissues, anatomical structures, or body parts. Healthcare professionals may view and analyze medical images to provide healthcare services to patients. Medical images may be stored on a database associated with a computing device and rendered on a user interface to be viewed by a healthcare professional.

### SUMMARY

Various implementations of systems, methods and devices within the scope of the appended claims each have several aspects, no single one of which is solely responsible for the desirable attributes described herein. Without limiting the scope of the appended claims, the description below describes some prominent features.

Details of one or more implementations of the subject matter described in this specification are set forth in the accompanying drawings and the description below. Other features, aspects, and advantages will become apparent from the description, the drawings, and the claims. Note that relative dimensions of the following figures may not be drawn to scale.

A computing system may prefetch exams to a local data store. The computing system can access the prefetched exam from the local data store to render the exams for viewing rather than retrieving the exams from a remote repository, which may significantly reduce the time required to render the exams for viewing. For example, a browser application executing on a computing system may access prefetched exams stored in the computing system hard drive and place them in the browser's cache to be accessed by a browser-based viewer program for rendering. For example, the browser-based viewer program may be accessible via a URL in the browser (e.g., www.thebestimageviewer.com) to provide a user interface configured to display and allow interactions with images. Rendering prefetched images may be much faster than rendering images that must be retrieved from a remote repository. Moreover, implementing a web-based viewer program to render exams, rather than a local viewer program on the computing system (e.g., installed as a standalone application), may reduce the computing system's processing requirements, storage requirements, and/or system architecture requirements. Accordingly, the systems and methods described herein provide for faster rendering using prefetched images that are accessible to a web-based viewer program rather than a locally installed viewer program, which can be expensive to install and/or require significant computer processing and/or memory. Additionally, use of a browser-based viewer program, even for viewing of locally cached images, provides a consistent user interface and user experience for all users. In some implementations, such as when exams have not been prefetched to a local data store, the computing system can access a remote repository which can retrieve and/or render the exams to provide to the computing system for viewing.

### BRIEF DESCRIPTION OF THE DRAWINGS

The following drawings and the associated descriptions are provided to illustrate implementations of the present disclosure and do not limit the scope of the claims. Aspects and many of the attendant advantages of this disclosure will become more readily appreciated as the same become better understood by reference to the following detailed description, when taken in conjunction with the accompanying drawings.

FIG. 1A is a block diagram illustrating an example computing system in communication with a repository via a network.

FIG. 1B is a block diagram illustrating an example communication data flow between a computing system and a repository.

FIG. 1C is a block diagram illustrating an example communication data flow between a computing system and a viewer program.

FIG. 2 is a flowchart illustrating an example process relating to rendering exams.

### DETAILED DESCRIPTION

Although certain implementations, embodiments, and examples are disclosed below, the inventive subject matter extends beyond the specifically disclosed implementations to other alternative implementations and/or uses and to modifications and equivalents thereof. Thus, the scope of the claims appended hereto is not limited by any of the particular implementations described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain implementations; however, the order of description should not be construed to imply that these operations are order dependent. Additionally, the structures, systems, and/or devices described herein may be embodied as integrated components or as separate components. For purposes of comparing various implementations, certain aspects and advantages of these implementations are described. Not necessarily all such aspects or advantages are achieved by any particular implementation. Thus, for example, various implementations may be carried out in a manner that achieves or optimizes one advantage or group of advantages as taught herein without necessarily achieving other aspects or advantages as may also be taught or suggested herein.

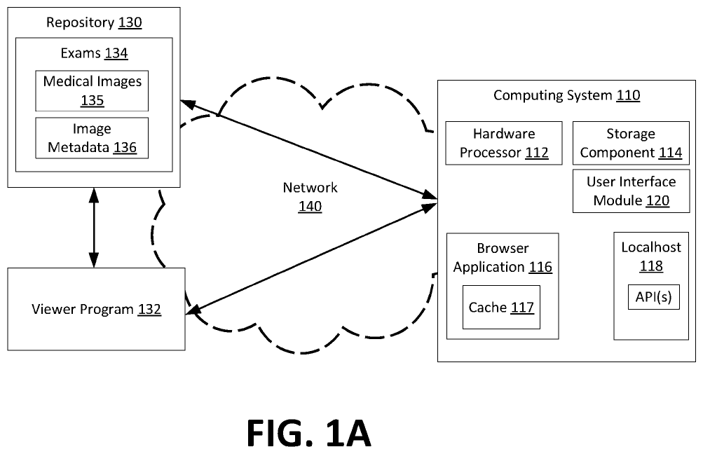

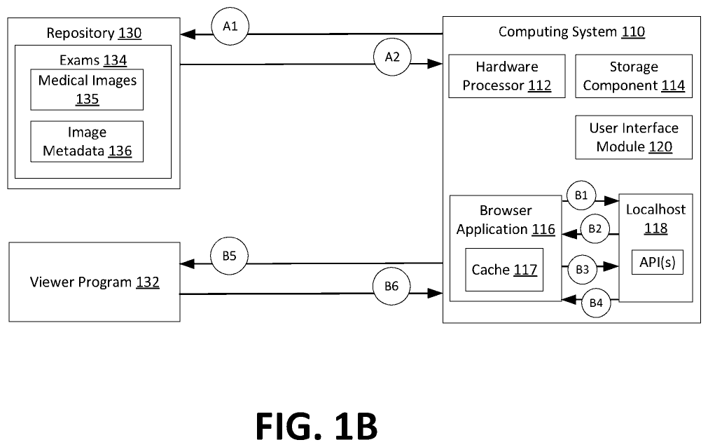

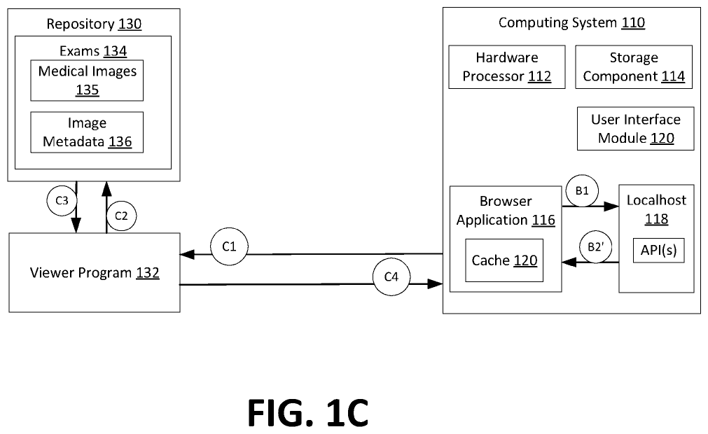

FIG. 1A is a block diagram illustrating an example computing system 110, repository 130, and viewer program 132 in communication via a network 140. The network 140 can include any one or more communications networks. The network 140 can include a plurality of computers configured to communicate with one another. The network 140 can include the Internet. The network 140 may be any combination of local area network (“LAN”) and/or a wide area network (“WAN”), or the like. Accordingly, various computing devices or systems, can communicate with one another directly or indirectly via any appropriate communications links and/or networks, such as network 140 (e.g., one or more communications links, one or more computer networks, one or more wired or wireless connections, the Internet, any combination of the foregoing, and/or the like).

The repository 130 may comprise one or more databases, file systems, cloud storage solutions, or hybrid storage systems, for example. These various storage systems and protocols may generally be referred to herein as a “database.” A database can be any data structure (and/or combinations of multiple data structures) for storing and/or organizing data, including, but not limited to, relational databases (e.g., Oracle databases, PostgreSQL databases, MySQL databases and the like), non-relational databases (e.g., NoSQL databases, and the like), in-memory databases, spreadsheets, as comma separated values (“CSV”) files, extensible markup language (“XML”) files, TeXT (“TXT”) files, flat files, spreadsheet files, and/or any other widely used or proprietary format for data storage. A database can include a storage device or system which can include any computer readable storage medium and/or device (or collection of data storage mediums and/or devices), including, but not limited to, one or more memory devices that store data, including without limitation, dynamic and/or static random-access memory (RAM), programmable read-only memory (PROM), erasable programmable read-only memory (EPROM), electrically erasable programmable read-only memory (EEPROM), optical disks (e.g., CD-ROM, DVD-ROM, etc.), magnetic disks (e.g., hard disks, floppy disks, etc.), memory circuits (e.g., solid state drives, random-access memory (RAM), etc.), and/or the like. Databases are typically stored in one or more data stores. Accordingly, each database referred to herein (e.g., in the description herein and/or the figures of the present application) can be understood as being stored in one or more data stores. Additionally, although the present disclosure may show or describe data as being stored in combined or separate databases, in various embodiments such data may be combined and/or separated in any appropriate way into one or more databases, one or more tables of one or more databases, and/or the like. A database may be hosted by a server. In some implementations, the repository 130 may include and/or be in communication with a hosted storage environment that includes a collection of physical data storage devices that may be remotely accessible and may be rapidly provisioned as needed (commonly referred to as “cloud” storage).

In some implementations, the repository 130 may comprise a Picture Archiving and Communication System (PACS). The viewer program 132 and/or repository 130 may be associated with and/or communicate with PACS to access exams stored in the PACS. In some implementations, the repository 130 may have retrieved the exams 134 from PACS. In some implementations, the viewer program 132 may be local to the repository 130. For example, the repository 130 and the viewer program 132 may be hosted by the same server and/or may be associated with the same database.

The viewer program 132 may include program instructions executable by a computing environment, such as by one or more hardware processors of a computing device, such as a user device or a cloud-based device or server. The viewer program 132 may include a browser-side implementation of a cloud-side backend. For example, some (or all) of the viewer program code may be executed within a web browser environment, such as by the browser's rendering engine, allowing for platform-independent functionality. In some implementations, the viewer program 132 communicates with a cloud-side backend via the internet, using web technologies such as HTML, CSS, and JavaScript. As discussed in further detail below, the viewer program 132 may use one or more APIs to interact with data and services outside of the browser.

The viewer program 132 can perform one or more operations relating to storing, accessing, managing, communicating, and/or displaying medical images. The viewer program 132 can generate user interface data for rendering medical images. The viewer program 132 may be a DICOM viewer program capable of handling, reading, rendering, transmitting, and/or manipulating DICOM images. The viewer program 132 may be hosted by a server. The viewer program 132 may be in communication with the repository 130, such as to access exams 134.

In some implementations, the viewer program 132 may provide tools to facilitate reading exams including viewing medical images, such as DICOM images. Example reading tools may include measurements tools for a user to measure anatomical features displayed in medical images on a user interface, annotation tools for a user to annotate medical images on a user interface, window and/or leveling tools such as for a user to adjust a contrast and/or brightness of an image, zoom and/or pan tools, 3D rendering tools to provide 3D reconstruction for volumetric data, rendering, and/or manipulation, multi-planar reconstruction tools to allow a user to view an image in different planes, such as axial, coronal, and/or sagittal, cross-reference tools to facilitate linking various images, image stacking tools for a user to overlay or superimpose images, image fusion tools, image filtering tools, or the like. The viewer program 132 can execute program instructions to perform tool operations. The viewer program 132 may be configured to access, read, analyze, and/or render image metadata, such as DICOM header information. The viewer program 132 may be configured to generate user interface data for rendering medical images, such as image slices of a multi-image series of images. The viewer program 132 may provide other features such as volume rendering, multi-planar reformatting, and/or 3D reconstruction of medical images.

The viewer program 132 can generate user interface data for modifying an appearance of a reading tool displayed within a user interface display. Modifying an appearance of a tool can include modifying a color, shading, location, size, etc. of a tool. In some implementations, modifying an appearance of a tool can including hiding a tool from view and/or not displaying a tool. The viewer program 132 can generate user interface data for modifying an appearance of a reading tool based on a tool's operational availability. A tool's operational availability may depend on whether the computing system 110 has access to exam(s) associated with the tool, such as whether the exams are prefetched. For example, a user may view a medical image of a patient's anatomy represented in 2D and may desire to view a 3D representation of the patient's anatomy. Executing a 3D reconstruction tool may provide the desired 3D representation but doing so would require access to relevant exams and the time required to access the relevant exams may differ greatly depending on whether the relevant exams are prefetched.

The viewer program 132 may modify a functionality of a reading tool, such as based on whether the exams are prefetched. For example, the viewer program 132 may apply one or more operations of a tool only to exams that are prefetched rather than to a fixed set of exams which may or may not be prefetched. Continuing with the example of executing a 3D reconstruction tool, the viewer program 132 may execute the tool on relevant exams that are prefetched without executing the tool on relevant exams that are not prefetched, which may result in a partial 3D reconstruction. This may advantageously provide the user with a rapid partial 3D reconstruction (e.g., performed only on prefetched exams) rather than forcing the user to wait for the tool to execute a full 3D reconstruction (e.g., on prefetched and non-prefetched exams). Other example reading tools are discussed herein whose operations may also be modified based on whether associated exams are prefetched. Advantageously, modifying an appearance of tools in a user interface and/or modifying operational functionality of tools (e.g., based on whether associated exams are prefetched locally) may improve a system performance by preventing a user and/or the viewer program 132 from performing tool operations that would require long amounts of time to access, retrieve, and/or render exams from a remote repository.

The repository 130 may store exams 134. The exams 134 can include one or more exams. An exam 134 can include one or more medical images 135, such as a single image or multiple images (e.g., from 2-1000 or more), or other data representing images, such as a 3D imaging volume that may be used to generate image slices at various angles and orientations. The medical images 135 may have been generated according using one or more imaging modalities, such as computed tomography (CT), magnetic resonance imaging (MRI), spectroscopy, endoscopy, mammography, positron emission tomography (PET), X-ray, ultrasound, digital radiography, computed radiography, and the like. The medical images 135 can include one or more types of images such as contrast or non-contrast images. The medical images 135 may comprise a series of images generated from a single imaging operation, such as a single scan. Images in a series of images may be referred to as slices. The medical images 135 can include Digital Imaging and Communications in Medicine (DICOM) images. The medical images 135 can include non-DICOM images. The medical images 135 can include one or more videos.

An exam 134 can include image metadata 136 that accompanies and/or is otherwise associated with a medical image 135. In general, image metadata 136 includes information about a medical image 135, such as imaging modality, imaging date, patient information, etc. For example, patient information included in metadata 136 may include patient demographics, medical history, or the like. In some embodiments, image metadata 136 may be stored in a DICOM header associated with a medical image. Image metadata 136 may be smaller in size (e.g., occupy less space in memory) than an associated medical image 135. Image metadata 136 can be communicated, accessed, retrieved, received, and/or stored, separately from medical images 135.

The computing system 110 may comprise one or more computing devices. In some implementations, one or more aspects of the computing system 110 may be implemented on a user device such as a computer, a laptop, a smartphone, a tablet, a mobile computing device, or the like. The computing system 110 may comprise and/or be referred to as a “client device” and/or a “local device”. A user, which may be a medical professional, such as a radiologist, referring doctor, hospital doctor or technician, etc. may interact with the computing system 110. In some implementations, the computing system 110 may be in communication with and/or hosted by one or more servers.

The computing system 110 can include one or more hardware processors 112 configured to execute program instructions to cause the computing system 110 to perform one or more operations. The hardware processor 112 can be configured, among other things, to process data, execute instructions to perform one or more functions, and/or control the operation of the computing system 110. The hardware processor 112 can execute instructions to perform functions related to storing and/or transmitting data. The hardware processor 112 can perform operations related to rendering exams for reading by a user.

The computing system 110 may access one or more APIs that allows more direct and/or secure communications with the viewer program 132 and/or repository 130. An API can facilitate communication through a specified channel enabling exchange of data and commands in a structured and secured manner. An API may comprise a set of defined communication protocols. An API may define a data format or data type for arguments (e.g., inputs) and responses (e.g., outputs). In some implementations, a plurality of APIs may be chained together with the response of one forming the arguments of another. An API can include one or more of a SOAP API, RPC API, Websocket API, REST API, Web API, or Web Service API. An API can include a private or public API. In some implementations, the computing system 110 may implement separate APIs to communicate with the viewer program 132 and the repository 130. In some embodiments, the viewer program 132 communicates with the repository 130, which may be stored at a different location than the viewer program 132 and/or associated with different security protocols, via another API. Other configurations may utilize other arrangements of APIs to facilitate communication between the various components of FIG. 1A.

As shown in FIG. 1A, the computing system 110 can access one or more operations of the viewer program 132, such as with an API. For example, the hardware processor 112 can cause the viewer program 132 to execute viewer program instructions to perform one or more operations, which can include operations relating to rendering images, performing reading tool operations, manipulating metadata, or the like. The hardware processor 112 can generate an API call to the viewer program 132 to execute operations of the viewer program 132. As an example, the hardware processor 112 can generate an API call to the viewer program 132 to cause the viewer program 132 to render exams (e.g., medical images of an exam). As another example, the hardware processor 112 can generate an API call to the viewer program 132 to cause the viewer program to implement one or more reading tools on an exam. The computing system 110 may communicate with the repository such as to retrieve exams 134.

The storage component 114 can include any computer readable storage medium and/or device (or collection of data storage mediums and/or devices), including, but not limited to, one or more memory devices that store data, including without limitation, dynamic and/or static random-access memory (RAM), programmable read-only memory (PROM), erasable programmable read-only memory (EPROM), electrically erasable programmable read-only memory (EEPROM), optical disks (e.g., CD-ROM, DVD-ROM, etc.), magnetic disks (e.g., hard disks, floppy disks, etc.), memory circuits (e.g., solid state drives, random-access memory (RAM), etc.), and/or the like.

The storage component 114 can store data such as processed and/or unprocessed data, exams, medical images, or the like. In some implementations, the storage component 114 may store data accessed from the repository 130. The storage component 114 can store prefetched exams. A prefetched exam can include an exam (which may include medical image(s) and/or associated metadata) accessed from a remote database, such as repository 130, according to one or more precaching rules and/or requests. A prefetched exam may also be referred to as a precached exam.

In the example of FIG. 1A, the computing system 110 executes a browser application 116. For example, the hardware processor 112 may execute program instructions associated with the browser application 116 to implement operations of the browser application 116. The browser application 116 may facilitate the computing system 110 interacting with the viewer program 132. For example, the browser application 116 may facilitate communication with a server hosting the viewer program 132. The browser application 116 may comprise a web browser configured to facilitate accessing and/or interacting with the World Wide Web. The browser application 116 may use one or more APIs to access data sources and services outside of the browser. As discussed further below, the viewer program 132 may be loaded into the browser application 116 and configure the browser application 116 to access medical images that are stored on a local storage device via API calls to a localhost server. The browser application 116 may comprise and/or maintain a local cache 117 on the computing system 110. The cache 117 may be a temporary storage. In some implementations, at least a portion of the cache may be maintained by the storage component 114, such as by RAM and/or a local storage device such as a Hard Disk Drive (HDD), Solid-State Drive (SSD), Hybrid Drive (SSHD), Removable Storage Device, and/or an Optical Drive. The cache 117 may store (e.g., temporarily) data associated with the viewer program 132, such as data from a server hosting the viewer program 132.

The computing system 110 may include a local networking interface referred to as localhost 118. The localhost 118, which may comprise a local server application, utilizes a loopback network interface of the computing system 110. It may be assigned a network address, such as 127.0.0.1 for IPv4 or ::1 for IPv6, which is configured to enable intrasystem communication, allowing the computing system 110 to send and receive network traffic to itself. The localhost 118 can host one or more APIs that facilitate operations such as data retrieval and manipulation. The computing system 110 can interact with these localhost APIs through HTTP requests to their respective endpoints. An HTTP endpoint typically corresponds to a specific URI (Uniform Resource Identifier) that is associated with the localhost 118 and delineates the functionality of the API resource or the routes for accessing and manipulating data.

The user interface module 120 may generate user interface data for rendering one or more interactive graphical user interfaces. The user interface module can render images, such as medical images of exams, based on user interface data generated by and/or received from viewer program 132. A user interface can include one or more medical images. A user interface can include image metadata. A user interface can include one or more tools for reading exams, such as measurement tools and/or annotation tools. In some implementations, the user interface module 120 may modify an appearance of reading tools within a user interface based on a tool's operational availability. A tool's operational availability may depend on whether the computing system 110 has access to exam(s) associated with the tool, such as whether the exams are prefetched. The user interface module 120 may receive and/or process user inputs, such as user inputs received via an interactive graphical user interface. For example, a user may adjust one or more display preferences for viewing medical images.

FIG. 1B is a block diagram illustrating an example flow of data between computing system 110, viewer program 132, and repository 130. FIG. 1B includes circles labelled as A1-A2, and B1-B6, which illustrate example interactions and data that may be exchanged between computing system 110 and repository 130. In other implementations, the interactions and/or data may be ordered differently.

At interaction A1, the computing system 110 can communicate a request to the repository 130, such as via an API call. The request may comprise a request to prefetch an exam from the repository 130 to the storage component 114 of the computing system 110. The computing system 110 can prefetch exams at a time before a user will use read the exam (e.g., view medical images of the exam). A request for an exam can comprise a request for a current exam and/or one or more other relevant exam(s). A current exam may be an exam to be read by a user, such as a radiologist. A relevant exam may be an exam associated with a current exam, such as a prior exam of the same patient. The computing system 110 may identify relevant exams based on relevancy rules and/or exam characteristics. For example, an exam may be a relevant exam if medical images of the exam pertain to a same anatomical region of a patient as medical images of a current exam. A request for an exam can comprise a request for image metadata 136 and/or medical images 135, whether alone or in combination. The computing system 110 may generate the request in response to a user command, such as a user requesting images of a particular medical exam. Depending on the implementation, the computing system 110 may automatically request medical exams from the repository 130 according to rules or conditions (e.g., the system is “pull driven”). Alternatively, the repository 130 may determine whether to transmit data to the computing system 110 according to rules or conditions (e.g., the system is “push driven”). For example, the repository 130 may be configured to automatically apply precaching rules when new medical exams are available in the exams 134. Example rules for precaching exams in a push driven system and/or pull driven system are described below.
Network characteristics. Precaching rules may consider one or more network characteristics such as speed, latency, bandwidth, throughput, or the like. For example, the computing system 110 may initiate precaching exams when a network has a large bandwidth capable of rapidly transmitting data between the computing system 110 and the repository 130. In one example, the computing system 110 sends a ping (or other similar message) to the repository 130 to determine current reachability of the repository 130. For example, such a command may indicate round-trip time, packet loss, and/or network congestion that may be relevant in determining if, when, and/or to what extent, precaching should be performed. Precaching exams based on network speed, etc. may improve system performance by reducing processing times required for transmitting data over the network.
Location. Precaching rules may consider a location of a user expected to read the requested exam and/or a location of a device associated with the user and/or computing system 110. In some implementations, the computing system 110 may be in communication with a user device such as a user's mobile phone, smartwatch, laptop, vehicle, etc. The computing system 110 may receive location data from the user's device such as from a location service on the user device which can include GPS data and/or other location information. In some implementations, the computing system 110 can access location data of a device associated with the computing system 110, such as a device used by a user to read exams. The computing system 110 can receive location data from the device used to read exams, such as from a location service on the device, which can include GPS data, and/or other location information. Precaching medical exams based on location may improve system performance such as by preventing downloading exams at times when a user and/or device may not be in a location appropriate to read the exams and/or by optimizing downloading exams at times when a user is likely to read exams such as when a user's location is near a computing device used to read exams. Precaching based on user and/or device location may improve security by preventing downloading exams when a user and/or device is not in a private or secure location.
Data size, quantity, and/or type. Precaching rules may consider a size of the exam, a number of files associated with the exam, type of the exam, and/or other attributes associated with the medical exams. The size and/or quantity can refer to current exam(s) and/or relevant exam(s). In some implementations, the computing system 110 may determine whether to request image metadata (e.g., DICOM headers) and/or medical images based on analyzing what information is needed, such as medical images or image metadata. For example, the computing system 110 may generate a request for only image metadata rather than generating a request for an entire exam (including medical images). Retrieving image metadata may be faster than retrieving an entire exam. In some implementations, the computing system 110 may generate the request based an imaging modality associated with images of the exam and/or image type.
Predicted time to retrieve. Precaching rules may consider an expected time required to receive the exam from the repository 130. An expected time to receive an exam may be based on a data size associated with the exam. In some implementations, the computing system 110 may avoid generating a request for an exam if an expected time to retrieve the exam is less than a threshold, such as if the exam size is sufficiently small. Determining to not generate a request to prefetch an exam may reduce network traffic such as by reducing marginally beneficial data transmission (e.g., precaching exams of small data size).
Permissions. Precaching rules may consider one or more permissions associated with the computing system 110 (e.g., a device used by a user to read exams), a user accessing the computing system 110 to read exams, an organization associated with the computing system 110 and/or a reading user, or the like. For example, the computing system 110 may generate the request when a reading user and/or a device used by the user is in a secure location.
User-based factors. Precaching rules may consider one or more factors associated with a reading user. For example, the computing system 110 may generate a request based on a user's assignment to read an exam. As another example, the computing system 110 may generate the request based on a reading user's schedule accessed and/or maintained by the computing system 110.
Clinical circumstances. Precaching rules may consider conditions such as a condition of a patient associated with an exam, such as whether the patient is in a critical health condition, whether the patient is scheduled for an operation, a length of time the patient has waited for medical attention, a date and/or time associated with the patient's admission to and/or departure from a health care facility, etc.
Preferences. Precaching rules may consider user preferences and/or organization preferences. A user and/or organization administrator may select, update, and/or delete preferences.
Minimizing redundancy. Precaching rules may consider whether an exam is already stored within the storage component 114. For example, in response to one or more factors triggering generating a request for an exam, the computing system 110 may first access the storage component 114 to determine whether the exam is already locally present and can determine whether to prefetch the exam. Reducing and/or eliminating unnecessary requests for data can reduce network traffic, reduce processing requirements, and increase system speed and/or performance. In some implementations, the computing system 110 may analyze multiple users'activity who are interacting with the computing system 110 to determine whether multiple users are using and/or requesting certain exams. For example, the computing system 110 may determine that multiple users need and/or are requesting a certain exam and the computing system 110 may accordingly generate a single request for the exam (e.g., rather than generating multiple requests for the same exam). Analyzing user activity can reduce redundant request for data which may improve system speed and/or performance, reduce network traffic, reduce processing requirements, or the like.

At interaction A2, the computing system 110 receives a response from the repository 130, such as via an API channel. The response can comprise data such as one or more exams, image metadata, and/or medical image(s), such as medical exams (or portions of medical exams) matching precaching rules. The computing system 110 can store data received from the repository 130, such as prefetched exams, including image data and/or metadata. In some implementations, the computing system 110 may implement a pre-processing operation on prefetched image data to generate instructions for respective stored image data. The instructions can comprise instructions for the browser application 116 to access the prefetched image data, such as via a localhost API, thereby bypassing browser limitations on direct hard drive access.

The browser application 116 can access services on the localhost 118, for instance, via an HTTP endpoint provided by a localhost API. Specifically, the browser application 116 may construct an API request targeting the localhost's API service using an HTTP endpoint. During interaction B1, the browser application 116 (executing the viewer program 132) may engage with an endpoint designed for metadata within the API. This metadata endpoint typically uses a URI that specifies a route for retrieving or updating image metadata related to pre-fetched exams stored on the computing system 110. The browser application 116 may initiate the request (such as an API call) at interaction B1 following a user's command to display one or more images from an exam.

At interaction B2, the localhost 118 can return a response associated with a metadata endpoint. For example, a localhost API associated with a metadata endpoint can return a response comprising image metadata which may be formatted as a JSON format and which may not include image data (e.g., pixel data). In some implementations, a response associated with a metadata endpoint may comprise instructions relating to exam(s) associated with retrieved metadata and which may instruct the browser application 116 how to access image pixel data of the exams that are prefetched on the computing system 110, such as by using a localhost API. In some implementations, the instructions may comprise pre-processed instructions, such as generated by the computing system 110 during or following interaction A2. The response may comprise instructions for rendering images, such as an order for retrieving image data to the browser cache to render images. In some implementations, such as described at interaction B2′ of FIG. 1C, a response may not comprise metadata and/or may comprise an indication that one or more exams are not prefetched on the computing system 110. The client-side viewer program 132 executing in the browser application 116 may determine whether exams and/or images are prefetched on the computing system 110 based on a metadata endpoint response, such as whether a response comprises image metadata or not. Advantageously, the browser application 116 may rapidly determine whether exams and/or images are prefetched on the computing system 110 with a metadata endpoint such as by requesting and/or receiving image metadata which may be smaller in size and/or easier to locate in storage than image pixel data.

At interaction B3, the viewer program executing in the browser application 116 may interact with an image data endpoint. An image data endpoint may comprise a URL and/or URI that defines a path for accessing and/or manipulating image data, including pixel data, of prefetched exams that are stored (outside of the browser cache) on the computing system 110. The browser application 116 may generate the request (e.g., API call) at interaction B1 in response to a user request to view one or more images of an exam. In some implementations, the viewer program executing in the browser application 116 may access the image data endpoint (e.g., generate an API request) after interacting with a metadata endpoint, such as in response to receiving an API response comprising image metadata, instructions for retrieving pixel data, and/or an indication that one or more exams are prefetched on the computing system 110. The browser application 116 may generate the request at interaction B1 based on the response received at interaction B2, which may include pre-processed instructions. The browser application 116 may request image data of respective images based on an order of rendering the images which may be determined by a viewer hanging protocol. For example, a viewer hanging protocol may render images in an order based on anticipated rendering speed which may be based on image size, reading tool availability, whether the images are prefetched (e.g., stored on the computing system 110), or the like. In some implementations, the browser application 116 may not interact with the image data endpoint (e.g., at interactions B3 and/or B4) in response to determining that one or more images and/or exams are not prefetched on the computing system 110, which may be determined based on a response provided to the browser application 116 at interaction B2.

At interaction B4, the localhost 118 can return a response associated with an image data endpoint. For example, a localhost API associated with an image data endpoint can return a response comprising image pixel data of prefetched images on the computing system 110. Advantageously, a localhost API may increase the amount of access that the viewer program executing in the browser application 116 has to data stored in the computing system 110, such as by increasing access to a hard drive of the computing system 110. The client-side viewer program executing in the browser application 116 can allocate image pixel data to the cache 117 to be accessed and/or manipulated by the viewer program 132. The viewer program 132 executing in the browser application 116 can render images from image pixel data in the browser cache 117. For example, the client-side viewer program 132 may generate user interface data to render images locally.

In some implementations, if the cache 117 storage space limit is reached, the browser application 116 may purge (e.g., delete) image data from the cache 117, such as by order of size. For example, the browser application 116 may purge large image data, such as related series of images, from the cache 117 before smaller image data. When space becomes available in the cache 117, the browser application 116 can again iterate through any of interactions B1-B4 to restore image data to cache 117 that may have been previously purged. Advantageously, the browser application 116 may be able to execute localhost APIs to retrieve image data to restore to the cache 117 that was previously purged rapidly enough that a user may not notice the effects of the cache 117 ever having been purged. For example, the computing system 110 may operate with minimal delays and/or rendering times.

In some implementations, the client-side portion of the viewer program 132 that executes in the browser application 116 may be configured to render and/or display images from the browser cache without further interactions (e.g., subsequent to interaction B4) with the server-side portion of the viewer program 132. In some instances, user interface details and available tools may be dynamically adjusted based on communications with the server-side viewer program 132. Interactions B5 and B6 illustrate examples of such communications that may enable a more customized environment for medical images. At interaction B5, the computing system 110 can communicate a request, such as an API call, to the viewer program 132, such as to perform one or more operations of viewer program 132. The request may be associated with one or more exams (e.g., prefetched exams) stored in the storage component 114 of the computing system 110. For example, the request may comprise a request to render pixel data of medical images into an appropriate format for display in the browser, such as JPG, PNG, BMP, or the like. Thus, the request can include image data, such as pixel data, associated with medical images of an exam. The request can include rendering parameters for rendering exams in a user interface, which may be based on user preferences, system settings, user viewing device specifications and/or settings, etc. The request may comprise a request to perform one or more operations of reading tool(s) provided by the viewer program 132 and/or associated with an exam, such as a measurement tool or annotation tool. The request can include tool operation information. The computing system 110 may generate the request in response to a user command. The computing system 110 may generate the request automatically, such as in response to one or more conditions. The computing system 110 may generate the request in response to determining that one or more exams are stored in the storage component 114 and/or in response to moving image pixel data to the cache 117 of the browser application 116.

At interaction B6, the computing system 110 can receive a response from the repository 130. The response can comprise an API response. The response can comprise user interface data for rendering medical images. For example, the viewer program 132 may execute program instructions to generate user interface data (e.g., from image data and/or rendering parameters received from the computing system via an API) which can then be communicated to the computing system 110 for rendering images. The response can comprise program instructions and/or data related to performing reading tool operations. For example, the viewer program 132 may execute program instructions to perform reading tool operations in response to a request from the computing system 110.

The viewer program 132 can generate user interface data for modifying an appearance of a reading tool displayed within a user interface display based on a tool's operational availability, such as whether exams associated with the tool are prefetched. In some implementations the viewer program 132 may modify a functionality of a reading tool, such as based on whether exams associated with the tool are prefetched.

FIG. 1C is a block diagram illustrating another example flow of data between computing system 110, viewer program 132, and repository 130. FIG. 1C includes circles labelled as B1-B2′ and C1-C4, which illustrate example interactions and data that may be exchanged between computing system 110, viewer program 132, and repository 130. In other implementations, the interactions and/or data may be ordered differently.

At interaction B1, the browser application 116 (executing instruction of the viewer program) may interact with a metadata endpoint such as shown and/or described at FIG. 1B. At interaction B2′, the browser application 116 may receive a response which may not comprise metadata and/or may comprise an indication that one or more exams are not prefetched on the computing system 110. The browser application 116 may determine that exams and/or images are not prefetched on the computing system 110 based on the response at interaction B2′. Because the exams and/or images are not prefetched, the browser application 116 may not be able to retrieve corresponding image pixel data from the computing system 110 to provide to the viewer program 132, and the viewer program 132 may need to retrieve such image pixel data from another sources, such as the repository 130. In some implementations, the computing system 110 and/or viewer program 132 may prioritize rendering prefetched exams before rendering exams that are not prefetched. For example, the computing system may render prefetched exams before and/or during one or more of interactions C1-C4.

At interaction C1, the computing system 110 can initiate a request for the viewer program 132 to retrieve exams. The computing system 110 may generate the request in response to a user request to read an exam and/or in response to determining that one or more exams are not prefetched on the computing system 110. The request may comprise a request to render medical images of an exam.

At interaction C2, the viewer program 132 may access one or more exams 134 which may be stored in the repository 130 which may be remote to the viewer program 132. In some implementations, the exams 134 may be stored in in a data store local to the viewer program 132. At interaction C3, the viewer program 132 can receive one or more exams 134, including medical images (e.g., pixel data) and/or image metadata.

At interaction C4, the viewer program 132 can communicate data to the computing system 110. The data can include exams, medical images, and/or image metadata. The data can include image data, such as pixel data. The data can include user interface data for rendering medical images. The viewer program 132 may generate at least a portion of the data communicated to the computing system 110. For example, the viewer program 132 may execute program instructions to generate user interface data based on a least image data received from the repository 130. The computing system 110 can use data received from the viewer program 132 to render medical images for a user to read exams.

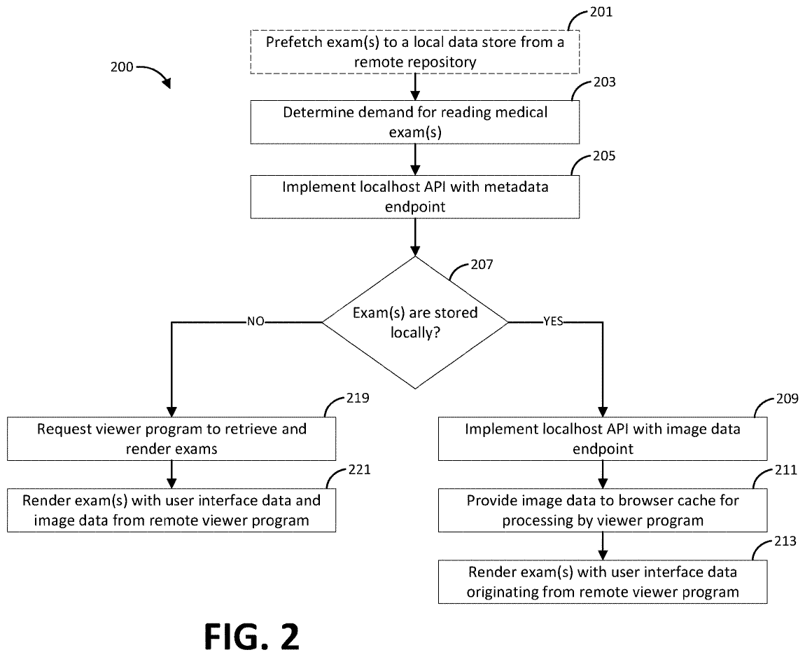

FIG. 2 is a flowchart illustrating an example process 200 for rendering medical images. This process, in full or parts, can be executed by one or more hardware processors, whether they're associated with a singular or multiple computing devices like computing system 110, and even devices in remote or wireless communication. The implementation may vary. For example, process 200 could be controlled by one or more hardware processors related to computing system 110, or can involve modifications like omitting blocks, adding blocks, and/or rearranging the order of execution of the blocks. Process 200 serves as an example and isn't intended to restrict the present disclosure.

At block 201, a computing device can optionally prefetch one or more exams to a local data store associated with the computing device from a remote repository. The computing device can prefetch the exams with an API. For example, the computing device may execute an API call to a remote repository for the exams and may receive an API response from the remote repository comprising the exams. The computing device can prefetch exams based on one or more conditions or precaching rules. Precaching exams to a local data store may reduce a time required to render medical images of the exams for a user to read the exams. In some implementations, the computing device may display to a user via a graphical user interface the exams that have been prefetched to a local data store. A user may be able to view which exams arc prefetched and select accordingly which exams the user would like to read at least because viewing which exams are prefetched will indicate to the user which exams may be faster to render for the user to read.

At block 203, the computing device can determine a demand for reading one or more medical exams, which may be referred to as current exam(s). For example, a user may desire to view one or more medical images associated with an exam. In some implementations, the computing device can determine the demand based on receiving a request from a user, such as via an interactive graphical user interface. In some implementations, the computing device may automatically determine the demand based one or more conditions, such as a schedule of a user, an order of the exams to be read, etc. In some implementations, the computing device may generate an order for reading the exams. For example, a plurality of exams may be assigned to a user to be read by the user. The computing device can generate a sequence in which the user is to read the exams. The computing device can present an option to read the exams and/or render the exams based on the order. The computing device can execute process 200, such as one or more of blocks 203-223, according to the order. The computing device can generate the order of exams based one or more factors such as whether an exam is stored locally, an exam size, an estimated time to retrieve an exam from a remote repository, and/or an estimated time to render an exam. In one example, the computing device may generate an order to present exams to a user by ordering locally stored exams before remotely stored exams and/or exams of small size before exams of large size. In some implementations, the computing device can generate the order based on additional factors such as a date an exam was generated, a date an exam was assigned to a user, a condition of a patient associated with an exam, or the like.

In some implementations, the computing device may identify relevant exams. The relevant exams may be associated with a current exam. The computing device may identify relevant exams based on at least relevancy rules. The computing device may identify relevant exams based on at least image metadata. In one example implementation, a relevant exam and a current exam may have a common frame of reference and/or may be associated with a same anatomical region. The computing device may identify the relevant exams based on a user input. For example, the computing device may identify all exams (e.g., relevant exams) that intersect, in one or more dimensions, a single location on a cross sectional image selected by a user via a user interface. These relevant exams may have a common frame of reference.

At block 205, the computing device may implement a localhost API using a metadata endpoint which may include initiating an API request to a metadata endpoint and receiving a response. The request may comprise retrieving image metadata from prefetched exams stored on the computing device. In some implementations, the corresponding response may comprise image metadata and/or instructions for retrieving image data such as pixel data of prefetched images stored on the computing device. In some implementations, the corresponding response may comprise an indication that the requested exams are not stored on the computing device.

At block 207, the computing device may determine whether exams (e.g., current exams and/or relevant exams) are stored locally on the computing device based on a localhost API response associated with a metadata endpoint. For example, the computing device may determine that exams are prefetched on the computing device if a localhost API response to a request for image metadata includes the image metadata. In some implementations, the computing device may display which exams are stored locally and/or which exams are not stored locally. In response to determining, that the exams are stored locally, the process may proceed to block 209. In response to determining that the exams are not stored locally, the process may proceed to block 219. In some implementations, the computing device may render prefetched exams before non-prefetched exams. For example, the computing device may perform one or more of blocks 209-213 before blocks 219-221. In some implementations, the computing device may perform block 219 subsequent to block 211. Advantageously, prioritizing prefetched exams may improve the speed at which a plurality of images is rendered and/or reduce a time that a user waits to view rendered images. For example, after rapidly rendering prefetched exams, the computing device may then implement rendering non-prefetched exams which may take longer to render than the prefetched exams. However, a user reading the exams may be able to view the rendered prefetched exams while the non-prefetched exams are in the process of rendering. Thus, prioritizing rendering exams based on rendering speed (e.g., whether they are prefetched) may reduce a user's time spent waiting for images to render in order to read the exams.

At block 209, the computing device can implement a localhost API using an image data endpoint. For example, the computing device can initiate an API call to retrieve image pixel data of images that are stored on the computing device and then, in turn, receive an API response comprising the image data.

At block 211, the image data may be stored in a browser cache for processing by the viewer program executing in the browser. In some implementations, the client-side remote viewer program may initiate a request to a cloud-side viewer program to perform one or more image rendering operations to render the images associated with the image data allocated to the browser cache. In some implementations, the rendering request to the cloud-side viewer program can include rendering parameters or display parameters.

At block 213, the viewer program executing in the browser of the computing device can render images of the prefetched exams with user interface data originating from the server-side remote viewer program and/or a webserver that provides the viewer program to the browser. For example, the computing device can receive user interface data that was generated by the remote viewer program and that is useable to render images corresponding to the image pixel data placed in the browser cache and stored locally on the computing device. The computing device can display the rendered exams on a screen. The screen may be remote to the computing device and/or integrated with the computing device.

If the exams are not stored locally at block 207, the method continues to block 219 where the computing device can initiate a request to retrieve the exam to a server-side viewer program or image repository. The exams may be stored on a remote repository that is remote to the computing device and/or to the viewer program. In some implementations, the viewer program in the browser sends opens an API channel with a remote repository to directly access the requested exam. The exams may then be stored in the browser cache and rendered in the viewer program directly from the browser cache. In some implementations, the exams may be stored on a repository that is local to the server-side viewer program, such as hosted by a same server and/or associated with a same database. In response to the request, the viewer program may access the exams from the repository and generate user interface data to render the exams.

At block 221, the viewer program executing in the browser of the computing device can render images of exams based on image pixel data that was stored in the browser cache in block 219. For example, the computing device can receive user interface data generated by the viewer program and useable to render images retrieved from the repository by the viewer program. As another example, the computing device can receive image pixel data that was retrieved from the repository by the viewer program. The computing device can display the rendered exams on a screen. The screen may be remote to the computing device and/or integrated with the computing device.

### Example Implementations

Examples of the implementations of the present disclosure can be described in view of the following example clauses. The features recited in the below example implementations can be combined with additional features disclosed herein. Furthermore, additional inventive combinations of features are disclosed herein, which are not specifically recited in the below example implementations, and which do not include the same features as the specific implementations below. For sake of brevity, the below example implementations do not identify every inventive aspect of this disclosure. The below example implementations are not intended to identify key features or essential features of any subject matter described herein. Any of the example clauses below, or any features of the example clauses, can be combined with any one or more other example clauses, or features of the example clauses or other features of the present disclosure.
Clause 1. A computerized method, performed by a computing system having one or more hardware computer processors and one or more non-transitory computer readable storage device storing software instructions executable by the computing system to perform the computerized method comprising: monitoring one or more cloud queues for image data; identifying, based on one or more prefetching parameters, image data corresponding to prefetching parameters; automatically initiating download of the matching image data; locally storing the matching image data from the one or more cloud queues; executing a pre-processing operation on the stored image data to generate viewer instructions for respective stored image data, the viewer instructions comprising instructions for accessing the image data stored locally, thereby bypassing browser limitations on direct access to local storage; executing, within a browser application running on the computing system, an image viewer program configured to display images; in response to a user request for image data, querying a local Application Programming Interface (API) for the requested image data from the local storage; receiving, if the requested image data is available locally through the local API, a JSON package including the viewer instructions for the requested image data; directing, based on the viewer instructions, the immediate preloading of a first image of the image data from the local storage into browser memory while concurrently initiating a background process to sequentially load subsequent images of the image data from the local storage into browser memory; and defaulting to retrieving the image data from the one or more cloud queues when not available locally.
Clause 2. The computerized method of clause 1, further comprising: managing local browser cache limitations by selectively purging image data from the browser cache and repulling image data to the browser cache to maintain a user experience of continuous image availability.
Clause 3. The computerized method of clause 1, wherein the pre-processing operation is executed via a locally installed service.
Clause 4. The computerized method of clause 1, wherein the viewer instructions indicate an API endpoint to a location of the local storage where the requested image data is stored.
Clause 5. The computerized method of clause 1 wherein the sequential loading of the subsequent images is based on viewer hanging protocols.
Clause 6. A cloud-based image data management system, comprising: a prefetching component configured to monitor cloud queues and automatically download image data; a local storage facility for storing downloaded image data; a local service operative to pre-process the image data and to produce instructions for a viewer program; a local API through which the viewer program requests and receives image data and associated instructions when such image data is available locally; and the viewer program configured to interact with the local API to receive image data and instructions for display of images of the downloaded image data.
Clause 7. A computing system comprising: a hardware computer processor; a non-transitory computer readable medium having software instructions stored thereon, the software instructions executable by the hardware computer processor to cause the computing system to perform operations comprising: access a remote repository to selectively prefetch one or more exams to a local data store from the remote repository based on one or more rules, the one or more exams comprising medical images; and responsive to a request from a user to view a first medical image: initiate a first localhost API request using a first endpoint to retrieve image metadata associated with the first medical image from the local data store; receive a first localhost API response comprising image metadata and instructions for retrieving image pixel data from the local data store; responsive to determining that the first medical image is stored in the local data store based on the first localhost API response, initiate a second localhost API request using a second endpoint to retrieve the image pixel data associated with the first medical image from the local data store; responsive to receiving a second localhost API response comprising the image pixel data, store the image pixel data in a browser cache accessible to a browser-based viewer program configured to render the first medical image; and on a display associated with the computing system, render the first medical image within the viewer program.
Clause 8. The computing system of clause 7 wherein the one or more rules relate to network characteristics including one or more of network speed, network latency, network bandwidth, and/or network throughput.
Clause 9. The computing system of clause 7 wherein the one or more rules relate to a location of the user.
Clause 10. The computing system of clause 7 wherein the hardware processor is further configured to: determine an operational functionality of one or more reading tools of the remote viewer program based on whether exams associated with the one or more reading tools are prefetched to the local data store.
Clause 11. The computing system of clause 10 wherein the hardware processor is further configured to: modify an appearance of the one or more reading tools in a user interface based on the operational functionality.
Clause 12. The computing system of clause 10 wherein the hardware processor is further configured to: modify one or more operations of the one or more reading tools based on the operational functionality.

### Additional Implementations

As used herein, “real-time” or “substantial real-time” may refer to events (e.g., receiving, processing, transmitting, displaying etc.) that occur at the same time or substantially the same time (e.g., neglecting any small delays such as those that are imperceptible and/or inconsequential to humans such as delays arising from electrical conduction or transmission). As a non-limiting example, “real-time” may refer to events that occur within a time frame of each other that is on the order of milliseconds, seconds, tens of seconds, or minutes. For example, “real-time” may refer to events that occur within a time frame of less than 1 minute, less than 30 seconds, less than 10 seconds, less than 1 second, less than 0.05 seconds, less than 0.01 seconds, less than 0.005 seconds, less than 0.001 seconds, etc. In some implementations, “real-time” may refer to events that occur at a same time as, or during, another event.

As used herein, “system,” “instrument,” “apparatus,” and “device” generally encompass both the hardware (for example, mechanical and electronic) and, in some implementations, associated software (for example, specialized computer programs for graphics control) components.

It is to be understood that not necessarily all objects or advantages may be achieved in accordance with any particular embodiment described herein. Thus, for example, those skilled in the art will recognize that certain implementations may be configured to operate in a manner that achieves or optimizes one advantage or group of advantages as taught herein without necessarily achieving other objects or advantages as may be taught or suggested herein.

Each of the processes, methods, and algorithms described in the preceding sections may be embodied in, and fully or partially automated by, code modules executed by one or more computer systems or computer processors including computer hardware. The code modules may be stored on any type of non-transitory computer-readable medium or computer storage device, such as hard drives, solid state memory, optical disc, and/or the like. The systems and modules may also be transmitted as generated data signals (for example, as part of a carrier wave or other analog or digital propagated signal) on a variety of computer-readable transmission mediums, including wireless-based and wired/cable-based mediums, and may take a variety of forms (for example, as part of a single or multiplexed analog signal, or as multiple discrete digital packets or frames). The processes and algorithms may be implemented partially or wholly in application-specific circuitry. The results of the disclosed processes and process steps may be stored, persistently or otherwise, in any type of non-transitory computer storage such as, for example, volatile or non-volatile storage.

Many other variations than those described herein will be apparent from this disclosure. For example, depending on the embodiment, certain acts, events, or functions of any of the algorithms described herein can be performed in a different sequence, can be added, merged, or left out altogether (for example, not all described acts or events are necessary for the practice of the algorithms). Moreover, in certain implementations, acts or events can be performed concurrently, for example, through multi-threaded processing, interrupt processing, or multiple processors or processor cores or on other parallel architectures, rather than sequentially. In addition, different tasks or processes can be performed by different machines and/or computing systems that can function together.

The various illustrative logical blocks, modules, and algorithm elements described in connection with the implementations disclosed herein can be implemented as electronic hardware, computer software, or combinations of both. To clearly illustrate this interchangeability of hardware and software, various illustrative components, blocks, modules, and elements have been described herein generally in terms of their functionality. Whether such functionality is implemented as hardware or software depends upon the particular application and design constraints imposed on the overall system. The described functionality can be implemented in varying ways for each particular application, but such implementation decisions should not be interpreted as causing a departure from the scope of the disclosure.

The various features and processes described herein may be used independently of one another, or may be combined in various ways. All possible combinations and sub-combinations are intended to fall within the scope of this disclosure. In addition, certain method or process blocks may be omitted in some implementations. The methods and processes described herein are also not limited to any particular sequence, and the blocks or states relating thereto can be performed in other sequences that are appropriate. For example, described blocks or states may be performed in an order other than that specifically disclosed, or multiple blocks or states may be combined in a single block or state. The example blocks or states may be performed in serial, in parallel, or in some other manner. Blocks or states may be added to or removed from the disclosed example implementations. The example systems and components described herein may be configured differently than described. For example, elements may be added to, removed from, or rearranged compared to the disclosed example implementations.

The various illustrative logical blocks and modules described in connection with the implementations disclosed herein can be implemented or performed by a machine, such as a general purpose processor, a digital signal processor (“DSP”), an application specific integrated circuit (“ASIC”), a field programmable gate array (“FPGA”) or other programmable logic device, discrete gate or transistor logic, discrete hardware components, or any combination thereof designed to perform the functions described herein. A general purpose processor can be a microprocessor, but in the alternative, the processor can be a controller, microcontroller, or state machine, combinations of the same, or the like. A processor can include electrical circuitry configured to process computer-executable instructions. In another embodiment, a processor includes an FPGA or other programmable devices that performs logic operations without processing computer-executable instructions. A processor can also be implemented as a combination of computing devices, for example, a combination of a DSP and a microprocessor, a plurality of microprocessors, one or more microprocessors in conjunction with a DSP core, or any other such configuration. Although described herein primarily with respect to digital technology, a processor may also include primarily analog components. For example, some, or all, of the signal processing algorithms described herein may be implemented in analog circuitry or mixed analog and digital circuitry. A computing environment can include any type of computer system, including, but not limited to, a computer system based on a microprocessor, a mainframe computer, a digital signal processor, a portable computing device, a device controller, or a computational engine within an appliance, to name a few.

The elements of a method, process, or algorithm described in connection with the implementations disclosed herein can be embodied directly in hardware, in a software module stored in one or more memory devices and executed by one or more processors, or in a combination of the two. A software module can reside in RAM memory, flash memory, ROM memory, EPROM memory, EEPROM memory, registers, hard disk, a removable disk, a CD-ROM, or any other form of non-transitory computer-readable storage medium, media, or physical computer storage known in the art. An example storage medium can be coupled to the processor such that the processor can read information from, and write information to, the storage medium. In the alternative, the storage medium can be integral to the processor. The storage medium can be volatile or nonvolatile. The processor and the storage medium can reside in an ASIC. The ASIC can reside in a user terminal. In the alternative, the processor and the storage medium can reside as discrete components in a user terminal.

Conditional language, such as, among others, “can,” “could,” “might,” or “may,” unless specifically stated otherwise, or otherwise understood within the context as used, is generally intended to convey that certain implementations include, while other implementations do not include, certain features, elements and/or steps. Thus, such conditional language is not generally intended to imply that features, elements and/or steps are in any way required for one or more implementations or that one or more implementations necessarily include logic for deciding, with or without user input or prompting, whether these features, elements and/or steps are included or are to be performed in any particular embodiment.

Disjunctive language such as the phrase “at least one of X, Y, or Z,” unless specifically stated otherwise, is otherwise understood with the context as used in general to present that an item, term, and so forth, may be either X, Y, or Z, or any combination thereof (for example, X, Y, and/or Z). Thus, such disjunctive language is not generally intended to, and should not, imply that certain implementations require at least one of X, at least one of Y, or at least one of Z to each be present.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount. As another example, in certain implementations, the terms “generally parallel” and “substantially parallel” refer to a value, amount, or characteristic that departs from exactly parallel by less than or equal to 10 degrees, 5 degrees, 3 degrees, or 1 degree. As another example, in certain implementations, the terms “generally perpendicular” and “substantially perpendicular” refer to a value, amount, or characteristic that departs from exactly perpendicular by less than or equal to 10 degrees, 5 degrees, 3 degrees, or 1 degree.

Any process descriptions, elements, or blocks in the flow diagrams described herein and/or depicted in the attached figures should be understood as potentially representing modules, segments, or portions of code which include one or more executable instructions for implementing specific logical functions or steps in the process. Alternate implementations are included within the scope of the implementations described herein in which elements or functions may be deleted, executed out of order from that shown or discussed, including substantially concurrently or in reverse order, depending on the functionality involved, as would be understood by those skilled in the art.

Unless otherwise explicitly stated, articles such as “a” or “an” should generally be interpreted to include one or more described items. Accordingly, phrases such as “a device configured to” are intended to include one or more recited devices. Such one or more recited devices can also be collectively configured to carry out the stated recitations. For example, “a processor configured to carry out recitations A, B and C” can include a first processor configured to carry out recitation A working in conjunction with a second processor configured to carry out recitations B and C.

All of the methods and processes described herein may be embodied in, and partially or fully automated via, software code modules executed by one or more general purpose computers. For example, the methods described herein may be performed by the computing system and/or any other suitable computing device. The methods may be executed on the computing devices in response to execution of software instructions or other executable code read from a tangible computer readable medium. A tangible computer readable medium is a data storage device that can store data that is readable by a computer system. Examples of computer readable mediums include read-only memory, random-access memory, other volatile or non-volatile memory devices, CD-ROMs, magnetic tape, flash drives, and optical data storage devices.

It should be emphasized that many variations and modifications may be made to the herein-described implementations, the elements of which are to be understood as being among other acceptable examples. All such modifications and variations are intended to be included herein within the scope of this disclosure. The section headings used herein are merely provided to enhance readability and are not intended to limit the scope of the implementations disclosed in a particular section to the features or elements disclosed in that section. The foregoing description details certain implementations. It will be appreciated, however, that no matter how detailed the foregoing appears in text, the systems and methods can be practiced in many ways. As is also stated herein, it should be noted that the use of particular terminology when describing certain features or aspects of the systems and methods should not be taken to imply that the terminology is being re-defined herein to be restricted to including any specific characteristics of the features or aspects of the systems and methods with which that terminology is associated.

Those of skill in the art would understand that information, messages, and signals may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

What is claimed is:

1. A computerized method, performed by a computing system having one or more hardware computer processors and one or more non-transitory computer readable storage device storing software instructions executable by the computing system to perform the computerized method comprising:
monitoring one or more cloud queues for image data;
identifying, based on one or more prefetching parameters, image data corresponding to prefetching parameters;
automatically initiating download of the matching image data;
locally storing the matching image data from the one or more cloud queues;
executing a pre-processing operation on the stored image data to generate viewer instructions for respective stored image data, the viewer instructions comprising instructions for accessing the image data stored locally, thereby bypassing browser limitations on direct access to local storage;
executing, within a browser application running on the computing system, an image viewer program configured to display images;
in response to a user request for image data, querying a local Application Programming Interface (API) for the requested image data from the local storage;
receiving, if the requested image data is available locally through the local API, a JSON package including the viewer instructions for the requested image data;
directing, based on the viewer instructions, the immediate preloading of a first image of the image data from the local storage into browser memory while concurrently initiating a background process to sequentially load subsequent images of the image data from the local storage into browser memory; and
defaulting to retrieving the image data from the one or more cloud queues when not available locally.

2. The computerized method of claim 1, further comprising: managing local browser cache limitations by selectively purging image data from the browser cache and repulling image data to the browser cache to maintain a user experience of continuous image availability.

3. The computerized method of claim 1, wherein the pre-processing operation is executed via a locally installed service.

4. The computerized method of claim 1, wherein the viewer instructions indicate an API endpoint to a location of the local storage where the requested image data is stored.

5. The computerized method of claim 1 wherein the sequential loading of the subsequent images is based on viewer hanging protocols.

6. A cloud-based image data management system, comprising:
a prefetching component configured to monitor cloud queues and automatically download image data;
a local storage facility for storing downloaded image data;
a local service operative to pre-process the image data and to produce instructions for a viewer program;
a local API through which the viewer program requests and receives image data and associated instructions when such image data is available locally; and
the viewer program configured to interact with the local API to receive image data and instructions for display of images of the downloaded image data.

7. A computing system comprising:
a hardware computer processor;
a non-transitory computer readable medium having software instructions stored thereon, the software instructions executable by the hardware computer processor to cause the computing system to perform operations comprising:
access a remote repository to selectively prefetch one or more exams to a local data store from the remote repository based on one or more rules, the one or more exams comprising medical images; and
responsive to a request from a user to view a first medical image:
initiate a first localhost API request using a first endpoint to retrieve image metadata associated with the first medical image from the local data store;
receive a first localhost API response comprising image metadata and instructions for retrieving image pixel data from the local data store;
responsive to determining that the first medical image is stored in the local data store based on the first localhost API response, initiate a second localhost API request using a second endpoint to retrieve the image pixel data associated with the first medical image from the local data store;
responsive to receiving a second localhost API response comprising the image pixel data, store the image pixel data in a browser cache accessible to a browser-based viewer program configured to render the first medical image; and
on a display associated with the computing system, render the first medical image within the viewer program.

8. The computing system of claim 7 wherein the one or more rules relate to network characteristics including one or more of network speed, network latency, network bandwidth, and/or network throughput.

9. The computing system of claim 7 wherein the one or more rules relate to a location of the user.

10. The computing system of claim 7 wherein the hardware processor is further configured to:
determine an operational functionality of one or more reading tools of the remote viewer program based on whether exams associated with the one or more reading tools are prefetched to the local data store.

11. The computing system of claim 10 wherein the hardware processor is further configured to:
modify an appearance of the one or more reading tools in a user interface based on the operational functionality.

12. The computing system of claim 10 wherein the hardware processor is further configured to:
modify one or more operations of the one or more reading tools based on the operational functionality.
